(19) United States
(12) Patent Application Publication
(10) Pub. No.: US 2009/0214382 A1
(43) Pub. Date: Aug. 27, 2009

(54) Methods of sterilizing biological mixtures using alpha-keto acids

(76) Inventors: Wilson Burgess, Clifton, VA (US); David Mann, Gaithersburg, MD (US); William Drohan, Germantown, MD (US); Shirley Miekka, Gaithersburg, MD (US)

Correspondence Address: MORGAN LEWIS & BOCKIUS LLP, 1111 PENNSYLVANIA AVENUE NW, WASHINGTON, DC, 20004, US

(21) Appl. No.: 11/579,497

(22) PCT Filed: May 5, 2005
(86) PCT No.: PCT/US2005/15518

§ 371 Date: Mar. 1, 2009

(51) Int. Cl. A61L 2/08

(52) U.S. Cl. 422/22

(57) ABSTRACT

Methods are disclosed for sterilizing biological materials to reduce the level of one or more biological contaminants or pathogens therein, such as viruses, bacterial (including inter- and intracellular bacteria, such as mycoplasmas, ureaplasmas, nanobacteria, chlamydia, rickettsias), yeasts, molds, fungi, single or multicellular parasites, and/or prions or similar agents responsible. These methods involve the use of alpha-keto acids in methods of sterilizing biological materials with irradiation.

### RELATED APPLICATIONS

The present application claims the benefit of U.S. Provisional Application 60/567,803 which is hereby incorporated by reference in its entirety.

### FIELD OF THE INVENTION

The present invention relates to methods for sterilizing biological materials to reduce the level of one or more biological contaminants or pathogens therein, such as viruses, bacteria (including inter- and intracellular bacteria, such as mycoplasmas, ureaplasmas, nanobacteria, chlamydia, rickettsias), yeasts, molds, fungi, single or multicellular parasites, and/or prions or similar agents responsible, alone or in combination, for TSEs. The present invention particularly relates to the use of alpha-keto acids in methods of sterilizing biological materials with irradiation.

### BACKGROUND OF THE RELATED ART

Many biological materials that are prepared for human, veterinary, diagnostic and/or experimental use may contain unwanted and potentially dangerous biological contaminants or pathogens, such as viruses, bacteria (including inter- and intracellular bacteria, such as mycoplasmas, ureaplasmas, nanobacteria, chlamydia, rickettsias), yeasts, molds, fungi, single or multicellular parasites, and/or prions or similar agents responsible, alone or in combination, for TSEs. Consequently, it is of utmost importance that any biological contaminant in the biological material be inactivated before the product is used. This is especially critical when the material is to be administered directly to a patient, for example in blood transfusions, blood factor replacement therapy, organ transplants and other forms of human therapy corrected or treated by intravenous, intramuscular or other forms of injection or introduction. This is also critical for the various biological materials that are prepared in media or via culture of cells or recombinant cells which contain various types of plasma and/or plasma derivatives or other biologic materials and which may contain prions, bacteria, viruses and other biological contaminants or pathogens.

Most procedures for producing biological materials have involved methods that screen or test the biological materials for one or more particular biological contaminants or pathogens rather than removal or inactivation of the contaminant(s) and/or pathogen(s) from the material. Materials that test positive for a biological contaminant or pathogen are merely not used. Examples of screening procedures include the testing for a particular virus in human blood from blood donors. Such procedures, however, are not always reliable and are not able to detect the presence of certain viruses, particularly in very low numbers. This reduces the value or certainty of the test in view of the consequences associated with a false negative result. False negative results can be life threatening in certain cases, for example in the case of Acquired Immune Deficiency Syndrome (AIDS). Furthermore, in some instances it can take weeks, if not months, to determine whether or not the material is contaminated. Moreover, to date, there is no reliable test or assay for identifying prions within a biological material that is suitable for screening out potential donors or infected material. This serves to heighten the need for an effective means of destroying prions within a biological material, while still retaining the desired activity of that material. Therefore, it would be desirable to apply techniques that would kill or inactivate biological contaminants and pathogens during and/or after manufacturing the biological material.

The importance of these techniques is apparent regardless of the source of the biological material. All living cells and multi-cellular organisms can be infected with viruses and other pathogens. Thus the products of unicellular natural or recombinant organisms or tissues carry a risk of pathogen contamination. In addition to the risk that the producing cells or cell cultures may be infected, the processing of these and other biological materials creates opportunities for environmental contamination. The risks of infection are more apparent for multicellular natural and recombinant organisms, such as transgenic animals. Interestingly, even products from species as different from humans as transgenic plants carry risks, both due to processing contamination as described above, and from environmental contamination in the growing facilities, which may be contaminated by pathogens from the environment or infected organisms that co-inhabit the facility along with the desired plants. For example, a crop of transgenic corn grown out of doors, could be expected to be exposed to rodents such as mice during the growing season. Mice can harbour serious human pathogens such as the frequently fatal Hanta virus. Since these animals would be undetectable in the growing crop, viruses shed by the animals could be carried into the transgenic material at harvest. Indeed, such rodents are notoriously difficult to control, and may gain access to a crop during sowing, growth, harvest or storage. Likewise, contamination from overflying or perching birds has the potential to transmit such serious pathogens as the causative agent for psittacosis. Thus any biological material, regardless of its source, may harbour serious pathogens that must be removed or inactivated prior to the administration of the material to a recipient.

In conducting experiments to determine the ability of technologies to inactivate viruses, the actual viruses of concern are seldom utilized. This is a result of safety concerns for the workers conducting the tests, and the difficulty and expense associated with the containment facilities and waste disposal. In their place, model viruses of the same family and class are used.

In general, it is acknowledged that the most difficult viruses to inactivate are those with an outer shell made up of proteins, and that among these, the most difficult to inactivate are those of the smallest size. This has been shown to be true for gamma irradiation and most other forms of radiation as these viruses' diminutive size is associated with a small genome. The magnitude of direct effects of radiation upon a molecule are directly proportional to the size of the molecule, that is the larger the target molecule, the greater the effect. As a corollary, it has been shown for gamma-irradiation that the smaller the viral genome, the higher the radiation dose required to inactive it.

Among the viruses of concern for both human and animal-derived biological materials, the smallest, and thus most difficult to inactivate, belong to the family of Parvoviruses and the slightly larger protein-coated Hepatitis virus. In humans, the Parvovirus B19, and Hepatitis A are the agents of concern. In porcine-derived materials, the smallest corresponding virus is Porcine Parvovirus. Since this virus is harmless to humans, it is frequently chosen as a model virus for the human B19 Parvovirus. The demonstration of inactivation of this model parvovirus is considered adequate proof that the method employed will kill human B19 virus and Hepatitis A, and by extension, that it will also kill the larger and less hardy viruses such as HIV, CMV, Hepatitis B and C and others.

More recent efforts have focussed on methods to remove or inactivate contaminants in the products. Such methods include heat treating, filtration and the addition of chemical inactivants or sensitizers to the product.

Heat treatment requires that the product be heated to approximately 60EC for about 70 hours which can be damaging to sensitive products. In some instances, heat inactivation can actually destroy 50% or more of the biological activity of the product. Filtration involves filtering the product in order to physically remove contaminants. Unfortunately, this method may also remove products that have a high molecular weight. Further, in certain cases, small viruses may not be removed by the filter.

The procedure of chemical sensitization involves the addition of noxious agents which bind to the DNA/RNA of the virus and which are activated either by UV or other radiation. This radiation produces reactive intermediates and/or free radicals which bind to the DNA/RNA of the virus, break the chemical bonds in the backbone of the DNA/RNA, and/or cross-link or complex it in such a way that the virus can no longer replicate. This procedure requires that unbound sensitizer is washed from products since the sensitizers are toxic, if not mutagenic or carcinogenic, and cannot be administered to a patient.

Irradiating a product with gamma radiation is another method of sterilizing a product. Gamma radiation is effective in destroying viruses and bacteria when given in high total doses (Keathly at al. (1993), “Is There Life After Irradiation? Part 2” BioPharm July-August, and Leitman (1989), Use of Blood Cell Irradiation in the Prevention of Post Transfusion Graft-vs-Host Disease” Transfusion Science 10:219-239). The published literature in this area, however, teaches that gamma radiation can be damaging to radiation sensitive products, such as blood, blood products, protein and protein-containing products. In particular, it has been shown that high radiation doses are injurious to red cells, platelets and granulocytes (Leitman). U.S. Pat. No. 4,620,908 discloses that protein products must be frozen prior to irradiation in order to maintain the viability of the protein product. This patent concludes that “[i]f the gamma irradiation were applied while the protein material was at, for example, ambient temperature, the material would be also completely destroyed, that is the activity of the material would be rendered so low as to be virtually ineffective”. Unfortunately, many sensitive biological materials, such as monoclonal antibodies (Mab), may lose viability and activity if subjected to freezing for irradiation purposes and then thawing prior to administration to a patient.

In view of the difficulties discussed above, there remains a need for methods of sterilizing compositions containing one or more biological materials that are effective for reducing the level of active biological contaminants or pathogens without an adverse effect on the material(s).

The above references are incorporated by reference herein where appropriate for appropriate teachings of additional or alternative details, features and/or technical background.

### SUMMARY OF THE INVENTION

The present invention encompasses a method for reducing the level of active biological contaminants or pathogens in a tissue, protein, plasma or serum sample, said method comprising adding to said tissue at least one alpha-keto acid stabilizer; and irradiating said tissue with a suitable dose of gamma radiation effective to reduce the level of active biological contaminants or pathogens in said tissue.

In one embodiment, the tissue is hard tissue and can be selected from bone, demineralized bone matrix, joints, femurs, femoral heads or teeth. In another embodiment, the tissue is soft tissue and can be selected from bone marrow, ligaments, tendons, nerves, skin grafts, heart valves, cartilage, corneas, arteries or veins. In yet another embodiment, tissue is a combination of hard and soft tissue.

In some embodiments of the invention, the sample to be irradiated is at a temperature below its freezing point during irradiation, and optionally, can be maintained in an inert atmosphere during irradiation including being under vacuum.

In some embodiments, the protein sample contains one or more proteins. Examples of proteins, include but are not limited to, an antibody, immunoglobulin, hormone, growth factor, anticoagulant, clotting factor or complement protein. Examples of clotting factors include, but are not limited to, Thrombin, Factor II, Factor V, Factor VII, Factor VIIa, Factor VIII, Factor IX, Factor X, Factor XIII, Factor XIIIa, Von Willebrand Factor, Fibrin or Fibrinogen. Imunoglobulins may be polyclonal or monoclonal immunoglobulins or mixtures thereof. Examples of immunoglobulins include, bu are not limited to, IgG, IgM, IgA, IgE or mixtures thereof.

In other embodiments, the protein is selected from the group consisting of protein C, protein S, alpha-1 anti-trypsin (alpha-1 protease inhibitor), heparin, insulin, butyl-cholinesterase, warfarin, streptokinase, tissue plasminogen activator (TPA), erythropoietin (EPO), urokinase, neupogen, antithrombin-3, alpha-glucosidase or albumin. The protein may be isolated or produced by recombinant or synthetic methods.

Samples containing serum to be irradiated by the methods of the invention include, but are not limited to, human serum.

In one embodiment of the invention, the concentration of the stabilizer is at least 20, 50, or 100 mM. Examples of alpha-keto acid stabilizers include, but are not limited to, Pyruvic acid. Alpha-keto butyrate, Sodium Pyruvate (Pyruvic acid sodium salt), Diacetyl (2,3-butanedione), Calcium Pyruvate (Pyruvic acid calcium salt), Glyoxylic acid (Oxoethanoic acid salt), Alpha-keto glutaric acid (sodium salt), Methyl pyruvate, Phenyl glyoxylate, Alpha-keto adipic acid, 2-keto-3-deoxyoctonate, 2-keto-D-gluconic acid, 2-ketonexanoic acid (alpha keto caproate), Alpha-keto isocaproic acid, 2-keto-4-methylpentanoic acid, 2-keto-3-methyl butytic acid, 4-methylthio-2-oxobutanoic acid, 3-methyl-2-oxovaleric acid, 2-ketosuccinate (oxaloacetate), and 2-ketopentenoic (alpha-ketovaleric acid). The invention also encompasses methods which employ a a combination of two or more stabilizers which is added to said tissue, protein sample or plasma or serum.

In yet other embodiments, the tissue, protein sample plasma or serum contains one or more residual solvents which may be water or an organic solvent. The level of residual solvent in the sample may be reduced prior to irradiation by any means acceptable in the art including lyophilization.

The invention also encompasses a composition produced by the methods of the invention. In one embodiment, the composition is a sterile composition.g

### DETAILED DESCRIPTION OF PREFERRED EMBODIMENTS

### Definitions

Unless defined otherwise, all technical and scientific terms used herein are intended to have the same meaning as is commonly understood by one of ordinary skill in the relevant art.

As used herein, the singular forms “a” or “an” or “the” include the plural reference unless the context clearly dictates otherwise.

As used herein, the term “biological material” is intended to mean any substance derived or obtained from a living organism. Illustrative examples of biological materials include, but are not limited to, the following: cells; tissues; blood or blood components; proteins, including recombinant and transgenic proteins, and proteinaceous materials; enzymes, including digestive enzymes, such as trypsin, chymotrypsin, alpha-glucosidase and iduronodate-2-sulfatase; immunoglobulins, including mono and polyimmunoglobulins; botanicals; food; and the like. Preferred examples of biological materials include, but are not limited to, the following: ligaments; tendons; nerves; bone, including demineralized bone matrix, grafts, joints, femurs, femoral heads, etc.; teeth; skin grafts; bone marrow, including bone marrow cell suspensions, whole or processed; heart valves; cartilage; corneas; arteries and veins; organs, including organs for transplantation, such as hearts, livers, lungs, kidneys, intestines, pancreas, limbs and digits; lipids; carbohydrates; collagen, including native, afibrillar, atelomeric, soluble and insoluble, recombinant and transgenic, both native sequence and modified; enzymes; chitin and its derivatives, including NO-carboxy chitosan (NOCC); stem cells, islet of Langerhans cells and other cells for transplantation, including genetically altered cells; red blood cells; white blood cells, including monocytes; and platelets.

As used herein, the term “sterilize” is intended to mean a reduction in the level of at least one active or potentially active biological contaminant or pathogen found in the biological material being treated according to the present invention, As used herein, the term “biological contaminant or pathogen” is intended to mean a contaminant or pathogen that, upon direct or indirect contact with a biological material, may have a deleterious effect on a biological material or upon a recipient thereof. Such biological contaminants or pathogens include the various viruses, bacteria (including inter- and intracellular bacteria, such as mycoplasmas, ureaplasmas, nanobacteria, chlamydia, rickettsias), yeasts, molds, fungi, single or multicellular parasites, and/or prions or similar agents responsible, alone or in combination, for TSEs known to those of skill in the art to generally be found in or infect biological materials. Examples of biological contaminants or pathogens include, but are not limited to, the following: viruses, such as human immunodeficiency viruses and other retroviruses, herpes viruses, filoviruses, circoviruses, paramyxoviruses, cytomegaloviruses, hepatitis viruses (including hepatitis A, B and C and variants thereof), pox viruses, toga viruses, Epstein-Barr viruses and parvoviruses; bacteria (including mycoplasmas, ureaplasmas, nanobacteria, chlamydia, rickettsias), such as Escherichia, Bacillus, Campylobacter, Streptococcus and Staphylococcus; parasites, such as Trypanosoma and malarial parasites, including Plasmodium species; yeasts; molds; and prions, or similar agents, responsible alone or in combination for TSE (transmissible spongiform encephalopathies), such as scrapie, kuru, BSE (bovine spongiform encephalopathy), CJD (Creutzfeldt-Jakob disease), Gerstmann-Straeussler-Scheinkler syndrome, and fatal familial insomnia. Further, as used herein, the term “active biological contaminant or pathogen” is intended to mean a biological contaminant or pathogen that is capable of causing a deleterious effect, either alone or in combination with another factor, such as a second biological contaminant or pathogen or a native protein (wild-type or mutant) or antibody, in the biological material and/or a recipient thereof.

As used herein, the term “stabilizer” is intended to mean a compound or material that, alone and/or in combination, reduces damage to the biological material being irradiated to a level that is insufficient to preclude the safe and effective use of the material. Illustrative examples of stabilizers that are suitable for use include, but are not limited to, the following, including structural analogs and derivatives thereof: antioxidants; free radical scavengers, including spin traps, such as tert-butyl-nitrosobutane (tNB), α-phenyl-tert-butylnitrone (PBN), 5,5-dimethylpyrroline-N-oxide (DMPO), tert-butylnitrosobenzene (BNB), α-(4-pyridyl-1-oxide)-N-tert-butylnitrone (4-POBN) and 3,5-dibromo-4-nitroso-benzenesulphonic acid (DBNBS); combination stabilizers, i.e., stabilizers which are effective at quenching both Type I and Type II photodynamic reactions; and ligands, ligand analogs, substrates, substrate analogs, modulators, modulator analogs, stereoisomers, inhibitors, and inhibitor analogs, such as heparin, that stabilize the molecule(s) to which they bind.

Examples of additional stabilizers include, but are not limited to, the following: fatty acids. including 6,8-dimercapto-octanoic acid (lipoic acid) and its derivatives and analogues (alpha, beta, dihydro, bisnor and tetranor lipoic acid), thioctic acid, 6,8-dimercapto-octanoic acid, dihydrolopoate (DL-6,8-dithioloctanoic acid methyl ester), lipoamide, bisonor methyl ester and tetranor-dihydrolipoic acid, omega-3 fatty acids, omega-6 fatty acids, omega-9 fatty acids, furan fatty acids, oleic, linoleic, linolenic, arachidonic, eicosapentaenoic (EPA), docosahexaenoic (DHA), and palmitic acids and their salts and derivatives; carotenes, including alpha-, beta-, and gamma-carotenes; xanthophylls; sucrose, polyhydric alcohols, such as glycerol, mannitol, inositol, and sorbitol; propylene glycol, polypropylene glycol, sugars, including derivatives and stereoisomers thereof, such as xylose, glucose, ribose, mannose, fructose, erythrosine, threose, idose, arabinose, lyxose, galactose, allose, altrose, gulose, talose, and trehalose; amino acids and derivatives thereof, including both D- and L-forms and mixtures thereof, such as arginine, lysine, alanine, valine, leucine, isoleucine, proline, phenylalanine, glycine, serine, threonine, tyrosine, asparagine, glutamine, aspartic acid, histidine, N-acetylcysteine (NAC), glutamic acid, tryptophan, sodium capryl N-acetyl tryptophan, and methionine; azides, such as sodium azide; uric acid and its derivatives, such as 1,3-dimethyluric acid and dimethylthiourea; allopurinol; thiols, such as glutathione and reduced glutathione and cysteine; trace elements, such selenium, chromium, and boron; vitamins, including their precursors and derivatives, such as vitamin A, vitamin C (including its derivatives and salts such as sodium ascorbate and palmitoyl ascorbic acid) and vitamin E (and its derivatives and salts such as alpha- beta- gamma- delta- epsilon- zeta- and eta-tocopherols, tocopherol acetate and alpha-tocotrienol); chromanol-alpha-C6; 6-hydroxy-2,5,7,8-tetramethylchroma-2 carboxylic acid (Trolox) and derivatives

Other stabilizers include extraneous proteins, such as gelatin and albumin; tris-3-methyl-1-phenyl-2-pyrazolin-5-one (MCI-186); citiolone; puercetin; chrysin; dimethyl sulfoxide (DMSO); piperazine diethanesulfonic acid (PIPES); imidazole; methoxypsoralen (MOPS); 1,2-dithiane-4,5-diol; reducing substances, such as butylated hydroxyanisole (BHA) and butylated hydroxytoluene (BHT); cholesterol, including derivatives and its various oxidized and reduced forms thereof, such as low density lipoprotein (LDL), high density lipoprotein (HDL), and very low density lipoprotein (VLDL); probucol; indole derivatives; thimerosal; lazaroid and tirilazad mesylate; proanthenols; proanthocyanidins; ammonium sulfate; Pegorgotein (PEG-SOD); N-tert-butyl-alpha-phenylnitrone (PBN); 4-hydroxy-2,2,6,6-tetramethylpiperidin-1-oxyl (Tempol); mixtures of ascorbate, urate and Trolox C (Asc/urate/Trolox C); and peptides of two or more amino acids, any of which may be either naturally occurring amino acids, i.e., L-amino acids, or non-naturally occurring amino acids, i.e., D-amino acids, and mixtures, derivatives, and analogs thereof, including, but not limited to, arginine, lysine, alanine, valine, leucine, isoleucine, proline, phenylalanine, glycine, histidine, glutamic acid, tryptophan (Trp), serine, threonine, tyrosine, asparagine, glutamine, aspartic acid, cysteine, methionine, and derivatives thereof, such as N-acetylcysteine (NAC) and sodium capryl N-acetyl tryptophan, as well as homologous dipeptide stabilizers (composed of two identical amino acids), including such naturally occurring amino acids, as Gly-Gly (glycylglycine) and Trp-Trp, and heterologous dipeptide stabilizers (composed of different amino acids), such as carnosine (β-alanyl-histidine), anserine (β-alanyl-methylhistidine), and Gly-Trp.

Additional stabilizers also include flavonoids/flavonols, such as diosmin, quercetin, rutin, silybin, silidianin, silicristin, silymarin, apigenin, apiin, chrysin, morin, isoflavone, flavoxate, gossypetin, myricetin, biacalein, kaempferol, curcumin, proanthocyanidin B2-3-O-gallate, epicatechin gallate, epigallocatechin gallate, epigallocatechin, gallic acid, epicatechin, dihydroquercetin, quercetin chalcone, 4,4′-dihydroxy-chalcone, isoliquiritigenin, phloretin, coumestrol, 4′,7-dihydroxy-flavanone, 4′,5-dihydroxy-flavone, 4′,6-dihydroxy-flavone, luteolin, galangin, equol, biochanin A, daidzein, formononetin, genistein, amentoflavone, bilobetin, taxifolin, delphinidin, malvidin, petunidin, pelargonidin, malonylapiin, pinosylvin, 3-methoxyapigenin, leucodelphinidin, dihydrokaempferol, apigenin 7-O-glucoside, pycnogenol, aminoflavone, purpurogallin fisetin, 2′,3′-dihydroxyflavone, 3-hydroxyflavone, 3′,4′-dihydroxyflavone, catechin, 7-flavonoxyacetic acid ethyl ester, catechin, hesperidin, and naringin. Examples also include, but are not limited to, single stabilizers or combinations of stabilizers that are effective at quenching both Type I and Type II photodynamic reactions, and volatile stabilizers, which can be applied as a gas and/or easily removed by evaporation, low pressure, and similar methods.

Additional preferred examples of stabilizers for use in the methods of the present invention include alpha-keto acids and their derivatives. Examples of alpha-keto acids include, but are not limited to, Pyruvic acid, Alpha-keto butyrate, Sodium Pyruvate (Pyruvic acid sodium salt), Diacetyl (2,3-butanedione), Calcium Pyruvate (Pyruvic acid calcium salt), Glyoxylic acid (Oxoethanoic acid salt), Alpha-keto glutaric acid (sodium salt), Methyl pyruvate, Phenyl glyoxylate, Alpha-keto adipic acid, 2-keto-3-deoxyoctonate, 2-keto-D-gluconic acid, 2-ketohexanoic acid (alpha keto caproate), Alpha-keto isocaproic acid, 2-keto-4-methylpentanoic acid, 2-keto-3-methyl butytic acid, 4-methylthio-2-oxobutanoic acid, 3-methyl-2-oxovaleric acid, 2-ketosuccinate (oxaloacetate), and 2-ketopentenoic (alpha-ketovaleric acid).

Additional examples of stabilizers for use in the methods of the present invention also include alpha-hydroxy acids and their derivatives. Preferred alpha-hydroxy include, but are not limited to, Lactic Acid, Sodium Lactate, Lactate combined with pyruvate, Lactate combined with ascorbate, 2-hydroxy butyric acid (various salts), 2-hydroxypentanoic acid, 2-hydroxyhexanoic acid, 2-hydroxyoctanoic acid, 2-hydroxydecanoic acid and 2-hydroxydodecanoic acid. Preferred stabilizers include Cu(II), Fe(III), Zn(II), Mn(II), Chromium or Aluminum (especially if combined with pyruvate), Calcium salts, Magnesium salts and Cobalt. Preferred stabilizers also include chelators. Examples of chelators include, but are not limited to, Bathocuproine (especially if combined with Cu(II)), DTPA, Deferoxamine, EDTA and Sodium citrate.

The stabilizers for use in the methods of the present invention can be used alone or in combination of two or more. Preferred combinations include, but are not limited to, Pyruvate+Ascorbate, Pyruvate+Gly-Gly, Pyruvate+Lactate, Pyruvate+Copper, Pyruvate+Ascorbate+Copper, Pyruvate+Carnosine, Pyruvate+Carnosine+Ascorbate, Pyruvate+Iron, Alphaketoglutarate+metals (various), Pyruvate+metals (various), Pyruvate+Copper+Bathocuproine, Pyruvate+Copper+DTPA,

As used herein, the term “blood components” is intended to mean one or more of the components that may be separated from whole blood and include, but are not limited to, the following: cellular blood components, such as red blood cells, white blood cells, and platelets; blood proteins, such as blood clotting factors, enzymes, albumin, plasminogen, fibrinogen, and immunoglobulins; and liquid blood components, such as plasma, plasma protein fraction (PPF), cryoprecipitate, plasma fractions, and plasma-containing compositions.

As used herein, the term “cellular blood component” is intended to mean one or more of the components of whole blood that comprises cells, such as red blood cells, white blood cells, stem cells, and platelets.

As used herein, the term “blood protein” is intended to mean one or more of the proteins that are normally found in whole blood. Illustrative examples of blood proteins found in mammals, including humans, include, but are not limited to, the following: coagulation proteins, both vitamin K-dependent, such as Factor VII and Factor IX, and non-vitamin K-dependent, such as Factor VIII and von Willebrands factor; albumin; lipoproteins, including high density lipoproteins (HDL), low density lipoproteins (LDL), and very low density lipoproteins (VLDL); complement proteins; globulins, such as immunoglobulins IgA, IgM, IgG and IgE; and the like. A preferred group of blood proteins includes Factor I (fibrinogen), Factor II (prothrombin), Factor III (tissue factor), Factor V (proaccelerin), Factor VI (accelerin), Factor VII (proconvertin, serum prothrombin conversion), Factor VIII (antihemophiliac factor A), Factor IX; (antihemophiliac factor B), Factor X (Stuart-Prower factor), Factor XI (plasma thromboplastin antecedent), Factor XII (Hageman factor), Factor XIII (protransglutamidase), von Willebrands factor (vWF), Factor Ia, Factor IIa, Factor IIIa, Factor Va, Factor VIa, Factor VIIa, Factor VIIIa, Factor IXa, Factor Xa, Factor XIa, Factor XIIa, and Factor XIIIa. Another preferred group of blood proteins includes proteins found inside red blood cells, such as hemoglobin and various growth factors, and derivatives of these proteins.

As used herein, the term “liquid blood component” is intended to mean one or more of the fluid, non-cellular components of whole blood, such as plasma (the fluid, non-cellular portion of the whole blood of humans or animals as found prior to coagulation) and serum (the fluid, non-cellular portion of the whole blood of humans or animals as found after coagulation).

As used herein, the term “a biologically compatible solution” is intended to mean a solution to which a biological material may be exposed, such as by being suspended or dissolved therein, and remain viable, i.e., retain its essential biological, pharmacological, and physiological characteristics. Such solutions may be of any suitable pH, tonicity, concentration and/or ionic strength.

As used herein, the term “a biologically compatible buffered solution” is intended to mean a biologically compatible solution having a pH and osmotic properties (e.g., tonicity, osmolality, and/or oncotic pressure) suitable for maintaining the integrity of the material(s) therein, including suitable for maintaining essential biological, pharmacological, and physiological characteristics of the material(s) therein. Suitable biologically compatible buffered solutions typically have a pH between about 2 and about 8.5, and are isotonic or only moderately hypotonic or hypertonic. Greater or lesser pH and/or tonicity may also be used in certain applications. The ionic strength of the solution may be high or low, but is typically similar to the environments in which the biological material is intended to be used. Biologically compatible buffered solutions are known and readily available to those of skill in the art.

As used herein, the term “residual solvent content” is intended to mean the amount or proportion of freely-available liquid in the biological material. Freely-available liquid means the liquid, such as water or an organic solvent (e.g., ethanol, isopropanol, polyethylene glycol, etc.), present in the biological material being sterilized that is not bound to or complexed with one or more of the non-liquid components of the biological material. Freely-available liquid includes intracellular water. The residual solvent contents related as water referenced herein refer to levels determined by the FDA approved, modified Karl Fischer method (Meyer and Boyd (1959), Analytical Chem. 31:215-219; May et al. (1982), J. Biol. Standardization, 10:249-259; Centers for Biologics Evaluation and Research, FDA, (1990) Docket No. 89D-0140, 83-93) or by near infrared spectroscopy. Quantitation of the residual levels of other solvents may be determined by means well known in the art, depending upon which solvent is employed. The proportion of residual solvent to solute may also be considered to be a reflection of the concentration of the solute within the solvent. When so expressed, the greater the concentration of the solute, the lower the amount of residual solvent.

As used herein, the term “protein sample” or “proteinaceous material” is intended to mean any material derived or obtained from a living organism that comprises at least one protein or peptide. A proteinaceous material may be a naturally occurring material, either in its native state or following processing/purification and/or derivatization, or an artificially produced material, produced by chemical synthesis or recombinant/transgenic technology and, optionally, process/purified and/or derivatized. Illustrative examples of proteinaceous materials include, but are not limited to, the following: proteins and peptides produced from cell culture; milk and other dairy products; ascites; hormones; growth factors; materials, including pharmaceuticals, extracted or isolated from animal tissue or plant matter, such as heparin, insulin, and inulin; plasma, including fresh, frozen and freeze-dried, and plasma protein fraction; fibrinogen and derivatives thereof, fibrin, fibrin I, fibrin II, soluble fibrin and fibrin monomer, and/or fibrin sealant products; whole blood; protein C; protein S; alpha-1 anti-trypsin (alpha-1 protease inhibitor); butyl-cholinesterase; anticoagulants, such as coumarin drugs (warfarin); streptokinase; tissue plasminogen activator (tPA); erythropoietin (EPO); urokinase; Neupogen™; anti-thrombin-3; alpha-galactosidase; iduronate-2-sulfatase; (fetal) bovine serum/horse serum; meat; immunoglobulins, including anti-sera, monoclonal antibodies, polyclonal antibodies, and genetically engineered or produced antibodies; albumin; alpha-globulins; beta-globulins; gamma-globulins; coagulation proteins; complement proteins; and interferons.

As used herein, the term “radiation” is intended to mean radiation of sufficient energy to sterilize at least some component of the irradiated biological material. Types of radiation include, but are not limited to, the following: (i) corpuscular (streams of subatomic particles such as neutrons, electrons, and/or protons); (ii) electromagnetic (originating in a varying electromagnetic field, such as radio waves, visible (both mono and polychromatic) and invisible light, infrared, ultraviolet radiation, x-radiation, and gamma rays and mixtures thereof); and (iii) sound and pressure waves. Such radiation is often described as either ionizing (capable of producing ions in irradiated materials) radiation, such as gamma rays, and non-ionizing radiation, such as visible light. The sources of such radiation may vary and, in general, the selection of a specific source of radiation is not critical provided that sufficient radiation is given in an appropriate time and at an appropriate rate to effect sterilization. In practice, gamma radiation is usually produced by isotopes of Cobalt or Cesium, while UV and X-rays are produced by machines that emit UV and X-radiation, respectively, and electrons are often used to sterilize materials in a method known as “E-beam” irradiation that involves their production via a machine. Visible light, both mono- and polychromatic, is produced by machines and may, in practice, be combined with invisible light, such as infrared and UV, that is produced by the same machine or a different machine.

As used herein, the term “to protect” is intended to mean to reduce any damage to the biological material being irradiated, that would otherwise result from the irradiation of that material, to a level that is insufficient to preclude the safe and effective use of the material following irradiation. In other words, a substance or process “protects” a biological material from radiation if the presence of that substance or carrying out that process results in less damage to the material from irradiation than in the absence of that substance or process. Thus, a biological material may be used safely and effectively after irradiation in the presence of a substance or following performance of a process that “protects” the material, but could not be used safely and effectively after irradiation under identical conditions but in the absence of that substance or the performance of that process.

As used herein, an “acceptable level” of damage may vary depending upon certain features of the particular method(s) of the present invention being employed, such as the nature and characteristics of the particular biological material and/or non-aqueous solvent(s) being used, and/or the intended use of the biological material being irradiated, and can be determined empirically by one skilled in the art. An “unacceptable level” of damage would therefore be a level of damage that would preclude the safe and effective use of the biological material being sterilized. The particular level of damage in a given biological material may be determined using any of the methods and techniques known to one skilled in the art.

As used herein, the term “constant,” with respect to the rate of irradiation, is intended to include any variation in the rate of irradiation that results from natural decay of the source material over the duration of the sterilization procedure.

As used herein, the term “not constant,” with respect to the rate of irradiation, is intended to mean that the variation in the rate of irradiation is greater than any variation in the rate of irradiation that results from natural decay of the source material over the duration of the sterilization procedure.

A first embodiment of the present invention is directed to a method for sterilizing a biological material that is sensitive to radiation comprising: (i) adding to a biological material at least one stabilizer in an amount effective to protect the biological material, wherein the at least one stabilizer includes at least one alpha-keto acid, or a salt or ester thereof; and (ii) irradiating the biological material with radiation at an effective rate for a time effective to sterilize the biological material.

A second embodiment of the present invention is directed to a method for sterilizing a biological material that is sensitive to radiation comprising: (i) adding to a biological material at least one stabilizer in an amount effective to protect the biological material, wherein the at least one stabilizer includes at least one alpha-keto acid, or a salt or ester thereof; and (ii) irradiating the biological material with radiation to a total dose effective to sterilize the biological material at a rate effective to sterilize the biological material and to protect the biological material from the radiation.

According to the methods of the present invention, at least one stabilizing process may be applied to the biological material prior to irradiating. Such stabilizing processes include: (a) reducing the residual solvent content of the biological material; (b) reducing the temperature of the biological material; (c) reducing the oxygen content of the biological material; (d) adjusting or maintaining the pH of the biological material; (e) adding to the biological material at least one aqueous solvent and combinations thereof. According to such preferred embodiments of the present invention, one or more stabilizing process(es) may be applied either before and/or after adding to the biological material at least one stabilizer in an amount effective to protect the biological material, wherein the at least one stabilizer includes at least one alpha-keto acid, or a salt or ester thereof.

According to other embodiments of the present invention, a stabilizer mixture is added prior to irradiation of the biological material with radiation, wherein the stabilizer mixture includes at least one alpha-keto acid. This stabilizer mixture is preferably added to the biological material in an amount that is effective to protect the biological material from the radiation. Suitable amounts of stabilizer mixture may vary depending upon certain features of the particular method(s) of the present invention being employed, such as the particular stabilizer mixture being used and/or the nature and characteristics of the particular biological material being irradiated and/or its intended use, and can be determined empirically by one skilled in the art. Preferably, the stabilizer mixtures includes at least one alpha-keto acid and at least one dipeptide stabilizer, such as gly-gly.

According to certain methods of the present invention, the residual solvent content of the biological material is reduced prior to irradiation of the biological material with radiation. The residual solvent content is preferably reduced to a level that is effective to protect the biological material from the radiation. Suitable levels of residual solvent content may vary depending upon certain features of the particular method(s) of the present invention being employed, such as the nature and characteristics of the particular biological material being irradiated and/or its intended use, and can be determined empirically by one skilled in the art. There may be biological materials for which it is desirable to maintain the residual solvent content to within a particular range, rather than a specific value.

When the solvent is water, and particularly when the biological material is in a solid phase, the residual solvent content is generally less than about 15%, typically less than about 10%, more typically less than about 9%, even more typically less than about 8%, usually less than about 5%, preferably less than about 3.0%, more preferably less than about 2.0%, even more preferably less than about 1.0%, still more preferably less than about 0.5%, still even more preferably less than about 0.2% and most preferably less than about 0.08%.

The solvent may preferably be a non-aqueous solvent, more preferably a non-aqueous solvent that is not prone to the formation of free-radicals upon irradiation, and most preferably a non-aqueous solvent that is not prone to the formation of free-radicals upon irradiation and that has little or no dissolved oxygen or other gas(es) that is (are) prone to the formation of free-radicals upon irradiation. Volatile non-aqueous solvents are particularly preferred, even more particularly preferred are non-aqueous solvents that are stabilizers, such as ethanol and acetone.

In certain embodiments of the present invention, the solvent may be a mixture of water and a non-aqueous solvent or solvents, such as ethanol and/or acetone. In such embodiments, the non-aqueous solvent(s) is preferably a non-aqueous solvent that is not prone to the formation of free-radicals upon irradiation, and most preferably a non-aqueous solvent that is not prone to the formation of free-radicals upon irradiation and that has little or no dissolved oxygen or other gas(es) that is (are) prone to the formation of free-radicals upon irradiation. Volatile non-aqueous solvents are particularly preferred, even more particularly preferred are non-aqueous solvents that are stabilizers, such as ethanol and acetone.

In another embodiment, when the residual solvent is water, the residual solvent content of a biological material is reduced by dissolving or suspending the biological material in a non-aqueous solvent that is capable of dissolving water. Preferably, such a non-aqueous solvent is not prone to the formation of free-radicals upon irradiation and has little or no dissolved oxygen or other gas(es) that is (are) prone to the formation of free-radicals upon irradiation.

When the biological material is in a liquid phase, reducing the residual solvent content may be accomplished by any of a number of means, such as by increasing the solute concentration. In this manner, the concentration of protein in the biological material dissolved within the solvent may be increased to generally at least about 0.5%, typically at least about 1%, usually at least about 5%, preferably at least about 10%, more preferably at least about 15%, even more preferably at least about 20%, still even more preferably at least about 25%, and most preferably at least about 50%.

In certain embodiments of the present invention, the residual solvent content of a particular biological material may be found to lie within a range, rather than at a specific point. Such a range for the preferred residual solvent content of a particular biological material may be determined empirically by one skilled in the art.

While not wishing to be bound by any theory of operability, it is believed that the reduction in residual solvent content reduces the degrees of freedom of the biological material, reduces the number of targets for free radical generation and may restrict the solubility of these free radicals. Similar results might therefore be achieved by lowering the temperature of the biological material below its eutectic point or below its freezing point, or by vitrification to likewise reduce the degrees of freedom of the biological material. These results may permit the use of a higher rate and/or dose of radiation than might otherwise be acceptable. Thus, the methods described herein may be performed at any temperature that doesn't result in unacceptable damage to the biological material, i.e., damage that would preclude the safe and effective use of the biological material. Preferably, the methods described herein are performed at ambient temperature or below ambient temperature, such as below the eutectic point or freezing point of the biological material being irradiated.

The residual solvent content of the biological material may be reduced by any of the methods and techniques known to those skilled in the art for reducing solvent from a biological material without producing an unacceptable level of damage to the biological material. Examples of such methods include, but are not limited to, lyophilization, evaporation, concentration, centrifugal concentration, vitrification and spray-drying.

A particular method for reducing the residual solvent content of a biological material is lyophilization. Another method for reducing the residual solvent content of a biological material is spray-drying. Yet another method for reducing the residual solvent content of a biological material is vitrification, which may be accomplished by any of the methods and techniques known to those skilled in the art, including the addition of solute and or additional solutes, such as sucrose, to raise the eutectic point of the biological material, followed by a gradual application of reduced pressure to the biological material in order to remove the residual solvent, such as water. The resulting glassy material will then have a reduced residual solvent content.

According to certain methods of the present invention, the biological material to be sterilized may be immobilized upon a solid surface by any means known and available to one skilled in the art. For example, the biological material to be sterilized may be present as a coating or surface on a biological or non-biological substrate.

The radiation employed in the methods of the present invention may be any radiation effective for the sterilization of the biological material being treated. The radiation may be corpuscular, including E-beam radiation. Preferably the radiation is electromagnetic radiation, including x-rays, infrared, visible light, UV light and mixtures of various wavelengths of electromagnetic radiation. In one embodiment, the form of radiation is gamma radiation.

According to the methods of the present invention, the biological material is irradiated with the radiation at a rate effective for the sterilization of the biological material, while not producing an unacceptable level of damage to that material. Suitable rates of irradiation may vary depending upon certain features of the methods of the present invention being employed, such as the nature and characteristics of the particular biological material being irradiated, the particular form of radiation involved and/or the particular biological contaminants or pathogens being inactivated. Suitable rates of irradiation can be determined empirically by one skilled in the art. Preferably, the rate of irradiation is constant for the duration of the sterilization procedure. When this is impractical or otherwise not desired, a variable or discontinuous irradiation may be utilized.

According to the methods of the present invention, the rate of irradiation may be optimized to produce the most advantageous combination of product recovery and time required to complete the operation. Both low (<3 kGy/hour) and high (>3 kGy/hour) rates may be utilized in the methods described herein to achieve such results. The rate of irradiation is preferably be selected to optimize the recovery of the biological material while still sterilizing the biological material. Although reducing the rate of irradiation may serve to decrease damage to the biological material, it will also result in longer irradiation times being required to achieve a particular desired total dose. A higher dose rate may therefore be preferred in certain circumstances, such as to minimize logistical issues and costs, and may be possible when used in accordance with the methods described herein for protecting a biological material from irradiation.

According to a particular embodiment of the present invention, the rate of irradiation is not more than about 3.0 kGy/hour, more preferably between about 0.1 kGy/hr and 3.0 kGy/hr, even more preferably between about 0.25 kGy/hr and 2.0 kGy/hour, still even more preferably between about 0.5 kGy/hr and 1.5 kGy/hr and most preferably between about 0.5 kGy/hr and 1.0 kGy/hr.

According to another particular embodiment of the present invention, the rate of irradiation is at least about 3.0 kGy/hr, more preferably at least about 6 kGy/hr, even more preferably at least about 16 kGy/hr, and even more preferably at least about 30 kGy/hr and most preferably at least about 45 kGy/hr or greater.

According to yet another particular embodiment of the present invention, the maximum acceptable rate of irradiation is inversely proportional to the molecular mass of the biological material being irradiated.

According to the methods of the present invention, the biological material to be sterilized is irradiated with the radiation for a time effective for the sterilization of the biological material. Combined with irradiation rate, the appropriate irradiation time results in the appropriate dose of irradiation being applied to the biological material. Suitable irradiation times may vary depending upon the particular form and rate of radiation involved and/or the nature and characteristics of the particular biological material being irradiated. Suitable irradiation times can be determined empirically by one skilled in the art.

According to the methods of the present invention, the biological material to be sterilized is irradiated with radiation up to a total dose effective for the sterilization of the biological material, while not producing an unacceptable level of damage to that material. Suitable total doses of radiation may vary depending upon certain features of the methods of the present invention being employed, such as the nature and characteristics of the particular biological material being irradiated, the particular form of radiation involved and/or the particular biological contaminants or pathogens being inactivated. Suitable total doses of radiation can be determined empirically by one skilled in the art. Preferably, the total dose of radiation is at least 25 kGy, more preferably at least 45 kGy, even more preferably at least 75 kGy, and still more preferably at least 100 kGy or greater, such as 150 kGy or 200 kGy or greater.

The particular geometry of the biological material being irradiated, such as the thickness and distance from the source of radiation, may be determined empirically by one skilled in the art. A preferred embodiment is a geometry that provides for an even rate of irradiation throughout the material. A particularly preferred embodiment is a geometry that results in a short path length for the radiation through the material, thus minimizing the differences in radiation dose between the front and back of the material. This may be further minimized in some preferred geometries, particularly those wherein the material has a constant radius about its axis that is perpendicular to the radiation source, by the utilization of a means of rotating the preparation about said axis,

Similarly, according to certain methods of the present invention, an effective package for containing the biological material during irradiation is one which combines stabilize under the influence of irradiation, and which minimizes the interactions between the package and the radiation, Preferred packages maintain a seal against the external environment before, during and post-irradiation, and are not reactive with the biological material within, nor do they produce chemicals that may interact with the material within. Particularly preferred examples include but are not limited to containers that comprise glasses stable when irradiated, stoppered with stoppers made of rubber that is relatively stable during radiation and liberates a minimal amount of compounds from within, and sealed with metal crimp seals of aluminum or other suitable materials with relatively low Z numbers. Suitable materials can be determined by measuring their physical performance, and the amount and type of reactive leachable compounds post-irradiation and by examining other characteristics known to be important to the containment of biological materials empirically by one skilled in the art.

According to certain methods of the present invention, an effective amount of at least one sensitizing compound may optionally be added to the biological material prior to irradiation, for example to enhance the effect of the irradiation on the biological contaminant(s) or pathogen(s) therein, while employing the methods described herein to minimize the deleterious effects of irradiation upon the biological material. Suitable sensitizers are known to those skilled in the art, and include psoralens and their derivatives and inactines and their derivatives.

According to the methods of the present invention, the irradiation of the biological material may occur at any temperature that is not deleterious to the biological material being sterilized. According to one embodiment, the biological material is irradiated at ambient temperature. According to an alternate embodiment, the biological material is irradiated at reduced temperature, i.e. a temperature below ambient temperature or lower, such as 0° C., −20° C., −40° C., −60° C., −78° C. or −196° C. According to this embodiment of the present invention, the biological material is preferably irradiated at or below the freezing or eutectic point of the biological material. The irradiation of the biological material occurs at a temperature that protects the material from radiation.

In certain embodiments of the present invention, the temperature at which irradiation is performed may be found to lie within a range, rather than at a specific point. Such a range for the preferred temperature for the irradiation of a particular biological material may be determined empirically by one skilled in the art.

According to the methods of the present invention, the irradiation of the biological material may occur at any pressure which is not deleterious to the biological material being sterilized. According to one preferred embodiment, the biological material is irradiated at elevated pressure. More preferably, the biological material is irradiated at elevated pressure due to the application of sound waves or the use of a volatile. While not wishing to be bound by any theory, the use of elevated pressure may enhance the effect of irradiation on the biological contaminant(s) or pathogen(s) and/or enhance the protection afforded by one or more stabilizers, and therefore allow the use of a lower total dose of radiation. Suitable pressures call be determined empirically by one skilled in the art.

Generally, according to the methods of the present invention, the pH of the biological material undergoing sterilization is about 7. In some embodiments of the present invention, however, the biological material may have a pH of less than 7, preferably less than or equal to 6, more preferably less than or equal to 5, even more preferably less than or equal to 4, and most preferably less than or equal to 3. In alternative embodiments of the present invention, the biological material may have a pH of greater than 7, preferably greater than or equal to 8, more preferably greater than or equal to 9, even more preferably greater than or equal to 10, and most preferably greater than or equal to 11. According to certain embodiments of the present invention, the pH of the material undergoing sterilization is at or near the isoelectric point(s) of one or more of the components of the biological material. Suitable pH levels can be determined empirically by one skilled in the art.

Similarly, according to the methods of the present invention, the irradiation of the biological material may occur under any atmosphere that is not deleterious to the biological material being treated. According to one preferred embodiment, the biological material is held in a low oxygen atmosphere or an inert atmosphere. When an inert atmosphere is employed, the atmosphere is preferably composed of a noble gas, such as helium or argon, more preferably a higher molecular weight noble gas, and most preferably argon. According to another preferred embodiment, the biological material is held under vacuum while being irradiated. According to a particularly preferred embodiment of the present invention, a biological material (lyophilized, liquid or frozen) is stored under vacuum or an inert atmosphere (preferably a noble gas, such as helium or argon, more preferably a higher molecular weight noble gas, and most preferably argon) prior to irradiation. According to an alternative preferred embodiment of the present invention, a liquid biological material is held under low pressure, to decrease the amount of gas, particularly oxygen, dissolved in the liquid, prior to irradiation, either with or without a prior step of solvent reduction, such as lyophilization. Such degassing may be performed using any of the methods known to one skilled in the art.

In another embodiment, where the biological material contains oxygen or other gases dissolved within or associated with it, the amount of these gases within or associated with the material may be reduced by any of the methods and techniques known and available to those skilled in the art, such as the controlled reduction of pressure within a container (rigid or flexible) holding the material to be treated or by placing the material in a container of approximately equal volume.

In certain embodiments of the present invention, when the biological material to be treated is a tissue, the stabilizer mixture is introduced according to any of the methods and techniques known and available to one skilled in the art, including soaking the tissue in a solution containing the stabilizers, preferably under pressure, at elevated temperature and/or in the presence of a penetration enhancer, such as dimethylsulfoxide. Other methods of introducing the stabilizer mixture into a tissue include, but are not limited to, applying a gas containing the stabilizers, preferably under pressure and/or at elevated temperature, injection of the stabilizers or a solution containing the stabilizers directly into the tissue, placing the tissue under reduced pressure and then introducing a gas or solution containing the stabilizers, dehydration of the tissue by means known to those skilled in the art, followed by re-hydration using a solution containing said stabilizer(s), and followed after irradiation, when desired, by subsequent dehydration with or without an additional re-hydration in a solution or solutions without said stabilizer(s), and combinations of two or more of these methods. One or more sensitizers may also be introduced into a tissue according to such methods.

It will be appreciated that the combination of one or more of the features described herein may be employed to further minimize undesirable effects upon the biological material caused by irradiation, while maintaining adequate effectiveness of the irradiation process on the biological contaminant(s) or pathogen(s). For example, in addition to the use of a stabilizer mixture, a particular biological material may also be lyophilized, held at a reduced temperature and kept under vacuum prior to irradiation to further minimize undesirable effects.

The sensitivity of a particular biological contaminant or pathogen to radiation is commonly calculated by determining the dose necessary to inactivate or kill all but 37% of the agent in a sample, which is known as the D₃₇ value. The desirable components of a biological material may also be considered to have a D₃₇ value equal to the dose of radiation required to eliminate all but 37% of their desirable biological and physiological characteristics.

In accordance with certain preferred methods of the present invention, the sterilization of a biological material is conducted under conditions that result in a decrease in the D₃₇ value of the biological contaminant or pathogen without a concomitant decrease in the D₃₇ value of the biological material. In accordance with other preferred methods of the present invention, the sterilization of a biological material is conducted under conditions that result in an increase in the D37 value of the biological material. In accordance with the most preferred methods of the present invention, the sterilization of a biological material is conducted under conditions that result in a decrease in the D37 value of the biological contaminant or pathogen and a concomitant increase in the D37 value of the biological material.

### EXAMPLES

The following examples are illustrative, but not limiting, of the present invention. Other suitable modifications and adaptations are of the variety normally encountered by those skilled in the art and are fully within the spirit and scope of the present invention. Unless otherwise noted, all irradiation was accomplished using a ⁶⁰Co source.

### Example 1

Purpose: To determine the effects of gamma irradiation on collagen in the presence or absence of various stablizers.

Procedure: Set up type I collagen from rat tail for irradiation in liquid form. Collagen was resuspended at 1 mg/ml in 5 mM HOAC in the presence of the following radioprotectants:

1. None

2. G-G 100 mM (Sigma)

3. Lactate 100 mM (Sigma)

4. Sodium pyruvate 100 mM (Fluka)

5. Sodium pyruvate 100 mM/G-G 100 mM

The samples were frozen at −72° C. or kept at ambient temperature. Frozen samples were irradiated to a total dose of about 52.3 kGy to about 55.7 kGy at a dose rate of about 2.19 kGy/hr to about 2.33 kGy/hr. Ambient temperature samples were irradiated to a total dose of about 53.1 kGy to about 57.5 kGy at a dose rate of about 2.16 kGy/hr to about 2.34 kGy/hr.

Collagen was coated with 0.4 mg/ml of collagen or irradiated collagen in a 96-well U-bottom plate with a volume of 100 μl/well in 20 mM HOAC. The samples were Incubated for about 30 minutes at room temperature. The plate was dried overnight in the tissue culture hood.

1. None ambient temperature

2. None −72° C.

3. 100 mM thiourea −72° C.

4. 100 mM GG −72° C.

5. 100 mM lactic acid −72° C.

6. 100 mM pyruvate −72° C.

7. 100 mM pyruvate/100 mM GG

The plate was washed with PBS 5× at room temperature. HFF cells at passage 19 were removed from the plate by trypsin EDTA. The cells were recovered for about 1 hr at about 37° C. The cells were then washed with assay medium (DMEM/PS 2% BSA 20 mM Hepes pH 7.37) twice. The plate was blocked for about 1 hr at about 37° C. HFF cells were plated at 50,000 cells/well in assay medium. Cells were incubated for about 30 minutes at about 37° C.

Cells were washed with assay medium twice. Added 100 μl of MTT reagent to each well at 0.5 mg/ml in DMEM phenol red free and incubated for three hours at 37° C. MTT reagent was removed and cells were solubilized with 0.1 ml of 0.04 N HCl in isopropanol. The cell extracts (volume 100 μl) were mixed and read at OD 570 and 690 nm in a microplatereader.

Results: Samples irradiated at ambient temperature showed about 10% cell adhesion compared with the non-irradiated collagen. Samples irradiated at −72° C. showed 92% recovery. About 95% cell adhesion was detected for collagen irradiated in the presence of 100 mM GG. There was greater than 100% recovery for collagen irradiated in the presence of 100 mM GG and 100 m pyruvate. Samples containing thiourea at 100 mM showed about 92% cell adhesion recovery and samples containing pyruvate alone showed about 89% cell adhesion recovery. For samples containing both pyruvate and GG, the recovery yield was about 94%.

### Example 2

Purpose: To determine the effect of pyruvate on the structural integrity of albumin during gamma irradiation.

Procedure: The Red Nile fluorescence emission spectra of albumin were recorded on a Perkin Elmer LS50B spectrofluorometer. The irradiated (90 mg/ml) solution of HSA was diluted with PBS to a concentration of 5 mg/ml. 5 μL of 500 μM Nile red were added to 500 μL of PPF solution to a final concentration of Nile red of 5 μM. Fluorescence emission spectra were recorded at 25° C. in the wavelength range of 560 nm to 800 nm with excitation at 550 nm.

For SE HPLC, a BIOSEP 3000 column was equilibrated with PBS (pH 7.0) and run at 1 ml/min. Samples (5 mg/ml) were loaded on the column at a volume of 20 ρl. For differential scanning calorimetry, the DSC thermograms were recorded on a VP-DSC in a temperature range of 10 to 100° C. at a scanning rate 1 deg/min. The partial heat capacity of protein solution was calculated using the molecular weight of albumin 66.4 kDa, the partial specific volume 0.74 cm³/g and a protein concentration of ˜2 mg/ml.

For SDS PAGE, electrophoresis under denatured reduced and non-reduced conditions was carried out on an Invitrogen/Novex system using pre-cast 4-20% SDS-gel strips. Protein samples were loaded at 3 μg, 5 μg, 8 μg, and 12 μg per well.

The ligand-binding ability of irradiated albumin was investigated using caprylate. The relative effect of the stabilizer on the formation of the albumin-caprylate complex was monitored by measuring albumin's thermal stability in heating experiments in a range of caprylate concentrations from 0 to 32 mM.

Results: SDS-PAGE and SE HPLC analysis show that there is no detectable destruction of polypeptide chain or inter-molecular cross-linking, which would compromise the mass integrity of albumin. The Nile red fluorescence emission spectra with albumin before and after gamma irradiation in the presence of 50 mM pyruvate show no difference. The quantitative identity of the spectra indicates that the dynamic flexibility of albumin structure, as one of the criteria of its integrity, is not altered by irradiation.

DSC thermograms of the irradiated albumin showed no difference in the position of peak maximum, which indicates that there are no major changes in the structural stability of protein following irradiation. The position of the peak (Tₘₐₓ=64.7° C.) and integrated area of irradiated albumin (ΔH=3.93 cal/g), which are directly associated with the integrity of intramolecular interactions stabilizing protein conformation, were in good agreement with unirradiated albumin (Tₘₐₓ=64.53° C., ΔH=3.94 cal/g).

The analysis of changes in monomer/dimer population of albumin following 50 kGy dose of gamma irradiation, reveals a negligible increase of dimer population by 0.6% with nearly 1% decrease in monomer population.

Binding of the caprylate leads to an increase in the thermal stability of albumin in an additive manner as a result of the formation of a protein-ligand complex. The stabilization effect by the ligand in the range of concentration from 0 to 16 mM (equal to 0-320 μmol per gram of albumin) is practically identical for the irradiated and non-irradiate samples, indicating no differences in the ligand binding affinity.

Removal of the pyruvate from gamma irradiated albumin solutions following irradiation demonstrated that the gamma treated albumin remains its native structural properties and ligand binding activity. 95% of the population of albumin molecules retained their native structure and ligand-binding activity. Results demonstrate quantitative improvement of albumin stability against gamma irradiation damage in the presence of pyruvate. The presence of 50 mM pyruvate in 100 mg/ml albumin solution increased protein resistance by 15%-18% from 75-78% readily found for the non-protected albumin.

Exposure of liquid albumin to 50 kGy dose of gamma irradition in the presence of pyruvate has no detectable effect on its structural properties. No denatured protein was produced by gamma irradiation. Slight changes in the DSC heat capacity profile revealed that 5-8% of the protein was destabilized, but preserved its native properties. The dynamic flexibility of the albumin molecule, which is important for the recognition and binding affinity to specific biological ligands, was not compromised by gamma irradiation. No changes in the binding affinity to caprylate, which would indicate changes in its general binding site, were detected in the gamma-irradiated albumin. The presence of pyruvate in albumin product following irradiation does not interfere with its ligand-binding properties.

### Example 3

Purpose: To determine if the gamma radiolysis of aqueous pyruvate solution does gives rise to any not normally expected pyruvate products/derivatives as well as any significant modification in protein which would compromise its stability and structural integrity.

Method: FTIR spectra were recorded using FTS 3000MX Mid-IR Excalibur spectrometer (DigiLab) combined with HATR (horizontal attenuated total reflection, PIKE) at ambient temperature. Typically 400 scans have been accumulated in the range 500-4000 cm⁻¹ averaged, and corrected for water absorbance. Liquid samples were measured on the ZeSe crystal with a liquid holder and cover to avoid liquid evaporation. Solid samples were measured directly on the ZeSe crystal with a sample press to ensure good contact with crystal.

UV absorption of samples was measured using Perkin Elmer UV Lambda 35 spectrophotometer in the 260 nm to 400 nm wavelength range with 1 cm quartz cuvettes.

Procedure: For the effect of gamma irradiation on pyruvate, 1 M sodium pyruvate in PBS (pH 7) buffer were irradiated at 50 kGy on dry ice. UV and IR spectra before and after irradiation were measured and compared. In addition, solid sodium pyruvate powders before and after irradiation was also compared by FTIR.

For the effect of gamma irradiation on PPF, the aqueous solution of PPF in presence of 50 mM of sodium pyruvate was irradiated to 50 kGy at dry ice temperature. The FTIR spectra before and after irradiation were compared along with the spectra of pure pyruvate.

Results: FTIR spectra of pyruvate before and after gamma irradiation showed no difference indicating that the irradiation did not give rise to any significant modification in pyruvate. UV spectra of the pyruvate also showed no difference in the samples before and after irradiation, which indicates that there are no additional chemical compounds with other than for pyruvate absorption properties present in the irradiated solution.

FTIR spectrum of pyruvate solid powder is different from that of the aqueous solution (FIG. 3). However there are no differences in the position and amplitude of vibrational bands, which can be associated with changes in the chemical structure of the pyruvate powder after treatment.

It is expected that gamma-radiolysis of aqueous solution of pyruvate should produce high level of CO₂ and sodium acetate. Differences in the FTIR spectra of pyruvate expected from this reaction were observed. FTIR spectra of products of this reaction generated via adding H₂O₂ are quantitatively different from the spectra of pyruvate. The change in the position and amplitude of vibrational bands following the decarboxylation of pyruvate clearly demonstrates that if this reaction occurs during gamma irradiation, the pyruvate and acetate level variances would easily detected by this method. The observation that the FTIR spectra of pyruvate before and after irradiation are practically identical indicates that no decarboxilation of pyruvate occur during irradiation. The irradiated pyruvate remains its native chemical structure.

Further quantitative analysis can be done using the vibrational band at 1174.9 cm⁻¹ which is specific for the acetate and is more sensitive to the differences than the other vibrational bands in FTIR spectra. FTIR spectra of PPF solution containing 50 mM pyruvate before and after gamma irradiation show no difference. The pyruvate- and albumin-associated vibrational bands are present in a region of the intact pyruvate and albumin indicating that no modifications in FTIR detectable components subsequent to gamma irradiation treatment.

The DSC demonstrates differences in thermograms of pyruvate before and after irradiation indicating that minor irradiatio-mediated modification in pyruvate sample may occur. However, level of this modification seems to be negligibly low (have to be quantitated). The difference appears as an ability of the irradiated pyruvate solution to undergo exothermic chemical reaction at elevated temperature what is not typical for the unirradiated pyruvate. The chemical nature of this reaction has to be determined.

Conclusions: The gamma irradiation of aqueous solution of sodium pyruvate did not give rise to any detectable modification in pyruvate. Data obtained indicate that gamma-irradiation of aqueous pyruvate solution (1 M) at total dose 50 kGy at dry ice temperature does not produces detactable amounts of acetate and CO₂ as a result of decarboxylation reaction. Chemical structure of pyruvate remains intact after this treatment. The data presented also provide some evidence to suggest that presence of pyruvate during irradiation in concentration range 50 mM-100 mM does not cause derivativization of albumin, which would affect its structural properties.

### Example 4

Purpose: To compare the amount of proteoglycans present in porcine heart valves irradiated to 50 kGy in the presence or absence of various stabilizers compared to unirradiated samples.

Methods: Made up 50 ml of PDMT (propylene glycol+DMSO+mannitol+trehalose) as follows: 16.16% (v/v) propylene glycol, 22.06% (v/v) DMSO, 2.73% (w/v) mannitol, 3.78% (w/v) trehalose and 60% (v/v) water. Also made up 50 ml of PDMT (as above)+50 mM pyruvate. Used same percentages as above for PDMT and added 0.2751 g pyruvate.

Thawed heart valves at room temperature and washed valves four times with 5 ml of PBS and a final time with water, shaking at RT in 15 ml conicals. The heart valves were dried and three valves from the same pig were placed in plastic bags along with 10 ml of the appropriate solution were added to each bag.
0 kGy-water
50 kGy-water
PDMT-50 kGy
PDMT-0 kGy
PDMT+50 mM Pyruvate-50 kGy
PDMT+50 mM pyruvate-0 kGy

Samples were either not irradiated or irradiated to a total dose of about 58.4 kGy to about 59.4 kGy at a dose rate of about 2.39 kGy/hr to about 2.43 kGy/hr on dry ice. Following irradiation, proteins were isolated from the heart valves for SDS-PAGE analysis.

Conclusions: for SDS-PAGE, the untreated 50 kGy samples showed less proteoglycans than the other samples. For samples irradiated to 50 kGy, the PDMT treated samples irradiated to showed more recovery of the proteoglycans as compared to the samples not containing PDMT. However, the PDMT+50 mM pyruvate treated 50 kGy samples maintained the upper bands better than the samples containing PDMT alone. Overall, there appeared to be less degradation of the PDMT+50 mM pyruvate treated 50 kGy samples than the PDMT 50 kGy samples. The readings for the stains-all assay for each sample varied, but all points fell within the 0 to 20 μg/mL range.

### Example 5

Purpose: To investigate the effects of gamma irradiation on porcine heart valves in the presence of a stabilizer mixture containing pyruvate or not containing pyruvate.

Methods: 1. Prepared PDMT as described above along with a PDMT solution containing 50 mM pyruvate. Removed heart valves from growth medium & rinsed in milliQ water. Placed three valves from the same pig in plastic bags. Added 10 ml of the following solutions to each of two bags (one to be irradiated, one not to be irradiated): Water, PDMT and PDMT+50 mM Pyruvate. Heat-sealed each bag and placed each group (irradiated/unirradiated) in a larger bag. Oscillated the sample bags gently in a water bath at 40° C. for 4 hours. Allowed heart valves to soak for an additional 20 hours at 4° C. Briefly rinsed the heart valves in PBS. Placed heart valves in vacuum-sealed bags and stored in at −80° C. until ready for irradiation. Allowed heart valves to thaw at room temperature & placed in 15 ml conicals. Washed heart valves 4× with PBS (5 mL). Washed heart valves a final time with milliQ water (5 ml). Placed heart valves in labeled 5 ml screw cap conicals. Added 5 ml of 10% neutral phosphate buffered formalin to each conical. Nutated overnight at room temperature (minimum 12 hours). Washed valves once with 5 mL of 70% ethanol after removing formalin. Stored in fresh 5 ml of 70% ethanol at room temperature until delivered. Samples were either irradiated on dry ice to a total dose of about 58.4 kGy to about 59.4 kGy at a dose rate of about 2.39 kGy/hr to about 2.43 kGy/hr. Samples were analyzed on slides using Movatt's Pentachrome Stain.

Results: Samples irradiated and containing PDMT+pyruvate showed the same if not better recovery of the heart valve tissue.

### Example 6

Purpose: To examiner the effects of gamma irradiation of collagen of porcine ACL (anterior crucial ligaments) treated in the presence or absence of various stabilizers and stabilizer mixtures.

Methods: Porcine ACL were prepared as described above and were irradiated under the conditions described below. ACL were irradiated on dry ice to a total dose of about 56.3 kGy to about 58.1 kGy at a dose rate of about 2.28 kGy/hr to about 2.36 kGy/hr and collagen extraction performed on samples followed by SDS-PAGE analysis.
0 kGy-milliQ water
50 kGy-milliQ water
50 kGy-PDMT
50 kGy-PDMT 50 mM pyruvate
50 kGy-PDMT+100 mM pyruvate
50 kGy-propylene glycol, DMSO+100 mM pyruvate
50 kGy-propylene glycol+100 mM pyruvate
50 kGy-100 mM pyruvate

Results: Samples containing pyruvate showed improved recovery as measured by SDS-PAGE compared to samples not containing pyruvate.

### Example 7

Purpose: To investigate the effect of gamma radiation on collagen from heart valves in the presence or absence of various stabilizers using a protease sensitivity assay and amino acid analysis (AAA).

Methods: Prepared solutions and processed heart valves as desribed above. Irradiated heart valves on dry ice to a total dose of about 58.4 kGy to about 59.4 kGy at a dose rate of about 2.39 kGy/hr to about 2.43 kGy/hr. Dried and prepared samples for HPLC according to standard protocols. Samples were run on the HPLC according to the standard protocols.

Conclusion: The irradiated samples containing stabilizers showed less hydroxyproline in the supernatant, which indicated less degradation of the collagen compared to samples not containing stabilizers.

### Example 8

Purpose: To compare the amount of collagen present in the irradiated ACL treated with pyruvate to the 0 kGy control and the PDMT 50 kGy control.

Methods: Prepared PDMT+20 mM, 50 mM, 100 mM pyruvate as described above. ACL were processed as described above and irradiated ACL on dry ice to a total dose of about 56.3 kGy to about 58.1 kGy at a dose rate of about 2.28 kGy/hr to about 2.36 kGy/hr. Protein samples from irradiated ACL were prepared as per standard protocols and SDS-PAGE analysis conducted on the protein sample.

Results: It appeared that there was less degradation of the collagen in the samples that contained PDMT+50 mM and PDMT+100 mM pyruvate. Pyruvate without PDMT also seemed to show less degradation.

### Example 9

Purpose: To examine the effects of gamma irradiation of porcine heart valves in the presence or absence of various concentrations of pyruvate (PDMT, PDMT+50 or 100 mM pyruvate, PD+100 mM pyruvate, P+100 mM pyruvate, and 100 mM pyruvate. Irradiated samples on dry ice to a total dose of about 56.3 kGy to about 58.1 kGy at a dose rate of about 2.28 kGy/hr to about 2.36 kGy/hr. Prepare and analyzed irradiated samples on the HPLC according to the standard protocol.

Results: Samples treated with solutions containing pyruvate showed better recovery than samples treated with the PDMT solution. Recovery increased with increasing concentration of pyruvate.

### Example 10

Purpose: To examine the effects of gamma irradiation on Dermalink™ (a fibronectin product) prepared at lab-scale in the presence or absence of pyruvate. To quantify any loss of gelatin binding capacity of irradiated fibronectin via BCA assay.

Methods: A stock solution of fibronectin was prepared to 10 mg/ml. Samples were freeze-dried according to manufacturer's recommendations. Samples were irradiated with gamma irradiation on dry ice to a total dose of about 53.3 kGy to about 59.0 kGy. Samples were reconstituted and loaded onto a gelatin resin columns (about 2 μg irradiated fibronectin per 4 ml of gelatin resin solution). The eluate from the resin column washes was dialyzed against Tris buffer for two hours and analyzed by SDS-PAGE.

Results: SDS-PAGE analysis showed a protective effect for irradiated samples treated with pyruvate. Samples treated with 50 mM pyruvate showed greater recovery of protein bands.

According to the BCA assay, about 95% of irradiated Dermalink™ treated with 50 mM pyruvate binds to the resin. The presence of sodium alginate (a component of the Dermalink™ product) during irradiation appears not to alter the stabilizing effects of pyruvate.

### Example 11

Purpose: To examine the effects of gamma irradiation on vWF multimers irradiated in the presence of ascorbate, pyruvate and combinations of pyruvate and CuSO₄.

Methods: Samples of vWF multimers were reconstituted and irradiated on dry ice at a dose rate of 2.28 to 2.39 kGy/hr for a total dose of 55.8 to 58.7 kGy. Samples were analyzed by agarose gel electrophoresis.

Results: 50 mM pyruvate or the combination of 50 mM pyruvate and 10 μM CuSO₄ provided the greatest stabilizing effect on higher molecular weight vWF multimer integrity for irradiated samples. Additionally, 50 mM sodium ascorbate provided some stabilizing effect on higher molecular weight vWF multimer integrity for irradiated samples.

### Example 12

Purpose: To examine the effects of gamma radiation on recombinant α-1-antitrypsin (rAAT) irradiated to about 50 kGy on dry ice in the presence or absence lactate; pyruvate; ascorbate; pyruvate and ascorbate; lactate and ascorbate; or lactate and pyruvate.

Methods: Samples of rAAT were prepared along with the indicated concentration of stabilizer and irradiated samples on dry ice at a dose rate of about 2.27 kGy/hr to about 2.41 kGy/hr to a total dose of about 30.9 kGy to about 32.9 kGy. Samples were analyzed by SDS-PAGE and also using API Elastase Inhibition Kinetic Assay as per standard protocols. Samples were also analyzed by size exclusion chromatography (SEC) as per standard procedures.

Results: The irradiated samples containing 50 nmM, 25 mM or 100 mM sodium pyruvate had anti-elastase activity recoveries of 95, 89 and 90%, respectively. The frozen, irradiated Arriva rAAT samples containing 50 mM lactate had anti-elastase activity recovery of 89% and the samples irradiated without any stabilizer had a recovery of 92%. However, the samples irradiated with lactate had a higher potency than samples not containing a stabilizer (370 U/ml vs. 355 U/ml).

The irradiated samples containing 25 mM, 50 mM or 100 mM sodium ascorbate had anti-elastase activity recoveries of 93%, 91% and 87%, respectively. The potencies of the samples irradiated with 25 mM, 50 mM and 100 mM ascorbate were about the same (356 U/ml, 358 U/ml and 351 U/ml, respectively).

The irradiated samples containing the combination of 50 mM ascorbate and 50 mM pyruvate showed the best anti-elastase activity recovery (101%). The samples irradiated with the combination of 50 mM ascorbate and lactate had anti-elastase activity of 91% and the samples irradiated with the combination of 50 mM lactate and pyruvate had anti-elastase activity of 92%.

According to SDS-PAGE analysis, samples containing sodium pyruvate showed a significant decrease in degradation. Ascorbate and lactic acid alone did seem to prevent degradation. The best results were obtained for samples containing 100 mM Pyruvate; ascorbate and pyruvate; or lactic acid & pyruvate.

Following SEC, the amount of recovered protein is measured by taking the area below the largest peak (referred to as the monomer). The monomer is compared to the total area. Total area includes both the multimer (small peak ahead of the monomer) and monomer only. A large total area, such as 25 mM Asc/30, may have been due to more protein in the sample.

According to size exclusion chromatography, samples with the best recovery contained pyruvate as a stabilizer. Samples containing 100 mM Pyruvate; ascorbate & pyruvate; or lactic acid & pyruvate showed the best recoveries.

### Example 13

Purpose: To examine the effects of gamma irradiation on thrombin irradiated in the presence of 50 mM sodium ascorbate, 50 mM sodium pyruvate or 10 μM CuSO₄.

Procedure: Samples were prepared with stabilizer and irradiated on dry ice at a dose rate of about 2.23 kGy/hr to about 2.28 kGy/hr to a total dose of about 52.3 kGy to about 53.5 kGy and analyzed by SDS-PAGE. The samples were also analyzed sample for thrombin clotting time (TCT) assay using an MLA Electra 1400C device.

Results: According to SDS-PAGE, 50 mM sodium pyruvate had the best protective effects on thrombin structure integrity. Pyruvate especially protected the higher molecular weight proteins. The combination of 50 mM pyruvate and 50 mM ascobate or 50 mM pyruvate and 10 μM CuSO₄ showed the same protective effects on thrombin structure integrity. Additionally, 50 mM sodium ascorbate showed stabilizing effects on thrombin structure integrity during irradiation. CuSO₄ alone showed no protective effects on thrombin structure integrity during irradiation.

Based on TCT assays, sodium ascorbate, sodium pyruvate, CuSO₄, and combinations of ascorbate/pyruvate and pyruvate/CuSO₄ had protective effects on the clotting activity of frozen bovine thrombin during irradiation. TCT assays indicated that the combinations of 50 mM ascorbate and 50 mM pyruvate had the best protective effects on frozen bovine thrombin clotting activity. Additionally, the combination of pyruvate/CuSO₄ showed better protective effects on frozen bovine thrombin than pyruvate or CuSO₄ used alone.

### Example 14

Purpose: To examine the effects of gamma radiation on freeze-dried and frozen rAAT irradiated in the presence of various stabilizers, including pyruvate.

Methods: Samples were either irradiated at a dose rate of about 2.37 kGy/hr to about 2.55 kGy/hr (total dose: about 32.4 kGy to about 34.9 kGy) or about 2.16 kGy/hr to about 2.31 kGy/hr (total dose: about 52.6 kGy to about 53.9 kGy). Samples were analyzed by the API Elastase Inhibition Kinetic Assay according to standard protocols. Samples were also analyzed by size exclusion chromatography (SEC) according to standard protocols.

Results: The irradiated, freeze-dried rAAT samples had an anti-elastase inhibition activity recovery of 80% when irradiated to a total dose of about 30 kGy and 71% when irradiated to a total dose of about 50 kGy on dry ice at ˜2.5 kGy/hr.

Samples of frozen rAAT containing 50 mM sodium pyruvate had an anti-elastase inhibition activity recovery of 96% when irradiated to a total dose of about 30 kGy and 89% when irradiated to a total dose of about 50 kGy, compared to unirradiated samples. Samples of frozen rAAT containing 100 mM sodium ascorbate had an anti-elastase inhibition activity recovery of 94% when irradiated to a total dose of about 30 kGy and 87% when irradiated to a total dose of about 50 kGy, compared to unirradiated samples. Samples of frozen rAAT containing no stabilizer had an anti-elastase inhibition activity recovery of 87% when irradiated to a total dose of about 30 kGy and 80% when irradiated to a total dose of about 50 kGy, compared to unirradiated samples. Samples of frozen rAAT containing 200 μM trolox or 100 mM Gly-Gly showed very little or no protective effects compared to unirradiated samples.

SEC analysis showed that samples containing 50 mM sodium pyruvate had 97.7% recovery when irradiated to a total dose of about 30 kGy and 96.4% recovery when irradiated to about 50 kGy. Samples containing 100 mM sodium ascorbate had 91.7% recovery when irradiated to a total dose of about 30 kGy and 87.7% when irradiated to a total dose of about 50 kGy. Samples containing no stabilizer had 88.0% recovery when irradiated to a total dose of about 30 kGy and 80.9% recovery when irradiated to a total dose of about 50 kGy.

### Example 15

Purpose: To examiner the effects of gamma irradiation on API (Prolastin™ freeze-dried human alpha-I protease inhibitor (API)) irradiated in the presence or absence of various stabilizers, including pyruvate.

Methods: Irradiated frozen and freeze-dried API samples on dry ice at a dose rate of about 2.05 kGy/hr to about 2.23 kGy/hr to a total dose of about 52.7 kGy to about 57.2 kGy. Samples were then analyzed using the API Elastase Inhibition Kinetic Assay and also by SDS-PAGE.

Results: The frozen Prolastin™ irradiated with 100 mM pyruvate had 82% anti-elastase activity recovery. The frozen Prolastin™ irradiated without stabilizer had 74% recovery. The frozen Prolastin irradiated with 100 mM lactate had 83% anti-elastase activity recovery. The frozen Prolastin™ irradiated with a combination of 50 mM lactate and 50 mM sodium pyruvate had 88% anti-elastase activity recovery. The frozen Prolastin™ irradiated with 100 mM ascorbate had 85% anti-elastase activity recovery. The recoveries API anti-elastase activity of Prolastin™ with Gly-Gly, Lys-Trp-Lys and combinations of Asc/Gly-Gly, Asc/Lys-Trp-Lys stabilizers were about 80 to 82%.

According to SDS-PAGE, irradiated Prolastin™ without stabilizers showed aggregation at 200-66 kDa and degradation at 60-37 kDa.The addition of stabilizers reduced aggregation. However, degradation still occured at 50 kGy. Prolastin™ with 50 mM Pyruvate showed the least amount of degradation. Samples containing lactate and pyruvate showed improvement compared to samples containing only lactate.

### Example 16

Purpose: To examine the effects of gamma irradiation on freeze-dried Factor VIII (FVIII) irradiated in the presence or absence of 50 mM, 100 mM or 200 mM sodium pyruvate

Methods: Irradiated reconstituted samples on dry ice at a dose rate of about 2.14 kGy/hr to about 2.39 kGy/hr to a total dose of about 54.5 kGy to about 60.8 kGy. Samples were then analyzed for clotting activity using standard protocols.

Results: The addition of pyruvate improved the recovery of FVIII activity approximately 20 to 30% over FVIII not containing pyruvate. Samples containing 50 mM pyruvate had better recovery than samples containing either 100 mM pyruvate or 200 mM pyruvate.

### Example 17

Purpose: To examine the effects of gamma radiation on bovine thrombin irradiated on dry ice to a total dose of about 50 kGy in the presence of sodium pyruvate, sodium ascorbate or CuSO₄.

Methods: Samples were irradiated on dry ice to a total dose of about 53.9 kGy to about 61.1 kGy at a dose rate of about 2.14 kGy/hr to about 2.46 kGy/hr. Samples were analyzed using SDS-PAGE and a Performed Thrombin Clotting Time (TCT) Assay using MLA electra 1400C device as per manufacturer's recommendations.

Results: According to the thrombin clotting time assay, samples irradiated in the presence of combinations of 25 mM Asc/25 mM Pyr or 25 mM Asc/25 mM Pyr/1 mM CuSO₄ showed the best clotting activity recoveries. SDS-PAGE analysis showed that samples irradiated in the presence of 50 mM sodium pyruvate had better recovery than samples irradiated in the presence of 50 mM ascorbate. This appeared to be more pronounced for the higher molecular weight proteins. Samples containing either the combination of 25 mM pyruvate/25 mM ascorbate or 50 mM pyruvate/10 μM CuSO₄ showed improved recovery compared to samples containing only 50 mM pyruvate. Samples containing the combination of 25 mM ascorbate/25 mM pyruvate/1 μM CuSO₄ showed the best recovery.

Having now fully described this invention, it will be understood to those of ordinary skill in the art that the methods of the present invention can be carried out with a wide and equivalent range of conditions, formulations, and other parameters without departing from the scope of the invention or any embodiments thereof.

All patents and publications cited herein are hereby fully incorporated by reference in their entirety. The citation of any publication is for its disclosure prior to the filing date and should not be construed as an admission that such publication is prior art or that the present invention is not entitled to antedate such publication by virtue of prior invention.

The foregoing embodiments and advantages are merely exemplary and are not to be construed as limiting the present invention. The present teachings can be readily applied to other types of apparatuses. The description of the present invention is intended to be illustrative, and not to limit the scope of the claims. Many alternatives, modifications, and variations will be apparent to those skilled in the art, In the claims, means-plus-function clauses are intended to cover the structures described herein as performing the recited function and not only structural equivalents but also equivalent structures.

1. A method for reducing the level of active biological contaminants or pathogens in a tissue, said method comprising:
(i) adding to said tissue at least one alpha-keto acid stabilizer; and
(ii) irradiating said tissue with a suitable dose of gamma radiation effective to reduce the level of active biological contaminants or pathogens in said tissue.

2-9. (canceled)

10. A method for reducing the level of active biological contaminants or pathogens in a protein sample, said method comprising:
(i) adding to said protein sample at least one alpha-keto acid stabilizer; and
(ii) irradiating said protein sample with a suitable dose of gamma radiation effective to reduce the level of active biological contaminants or pathogens in said protein sample.

11. The method of claim 10, wherein said protein sample is at a temperature below its freezing point during irradiation.

12. The method of claim 10, wherein the protein sample is maintained in an inert atmosphere during irradiation.

13. (canceled)

14. The method of claim 10, wherein the protein sample contains one or more proteins.

15. The method of claim 14, wherein the protein is an antibody, immunoglobulin, hormone, growth factor, anticoagulant, clotting factor or complement protein.

16. (canceled)

17. The method of claim 15, wherein the immunoglobulins are polyclonal or monoclonal immunoglobulins or mixtures thereof.

18. (canceled)

19. The method of claim 14, wherein the protein is selected from the group consisting of protein C, protein S, alpha-1 anti-trypsin (alpha-1 protease inhibitor), heparin, insulin, butyl-cholinesterase, warfarin, streptokinase, tissue plasminogen activator (TPA), erythropoietin (EPO), urokinase, neupogen, antithrombin-3, alpha-glucosidase or albumin.

20. (canceled)

21. A method for reducing the level of active biological contaminants or pathogens in plasma or serum, said method comprising:
(i) adding to said plasma or serum at least one alpha-keto acid stabilizer; and
(ii) irradiating said plasma or serum with a suitable dose of gamma radiation effective to reduce the level of active biological contaminants or pathogens in said plasma or serum.

22. The method of claim 21, wherein the serum is human serum.

23. The method of claim 21, wherein said plasma or serum is at a temperature below its freezing point during irradiation.

24. The method of claim 21, wherein the plasma or serum is maintained in an inert atmosphere during irradiation.

25. (canceled)

26. The method of claim 21, wherein the concentration of the alpha-keto acid stabilizer is at least 20 mM.

27. The method of claim 21, wherein the concentration of the alpha-keto acid stabilizer is at least 50 mM.

28. The method of claim 21, wherein the concentration of the alpha-keto acid stabilizer is at least 100 mM.

29. The method of claim 21, wherein the one or more alpha-keto acid stabilizers is selected from the group consisting of Pyruvic acid, Alpha-keto butyrate, Sodium Pyruvate (Pyruvic acid sodium salt), Diacetyl (2,3-butanedione), Calcium Pyruvate (Pyruvic acid calcium salt), Glyoxylic acid (Oxoethanoic acid salt), Alpha-keto glutaric acid (sodium salt), Methyl pyruvate, Phenyl glyoxylate, Alpha-keto adipic acid, 2-keto-3-deoxyoctonate, 2-keto-D-gluconic acid, 2-ketohexanoic acid (alpha keto caproate), Alpha-keto isocaproic acid, 2-keto-4-methylpentanoic acid, 2-keto-3-methyl butytic acid, 4-methylthio-2-oxobutanoic acid, 3-methyl-2-oxovaleric acid, 2-ketosuccinate (oxaloacetate), and 2-ketopentenoic (alpha-ketovaleric acid).

30. (canceled)

31. The method of claim 21, wherein a combination of two or more stabilizers is added to said tissue, protein sample or plasma or serum.

32. (canceled)

33. The method of claim 21, wherein the tissue, protein sample plasma or serum contains one or more residual solvents.

34. (canceled)

35. The method of claim 33, wherein the residual solvent is an organic solvent.

36. The method of claim 33, wherein the residual solvent content in the sample is reduced prior to irradiation.

37. (canceled)
